(19) United States
(12) Patent Application Publication
(10) Pub. No.: US 2025/0177058 A1
(43) Pub. Date: Jun. 5, 2025

(54) ADAPTER FOR TRACKABLE SURGICAL INSTRUMENTS

(71) Applicant: TrackX Technology, Inc., Hillsborough, NC (US)

(72) Inventors: David A. Skwerer, Raleigh, NC (US); Robert E. Isaacs, Chapel Hill, NC (US); John Souza, Monroe, NC (US); Alexander Culbert, Durham, NC (US)

(21) Appl. No.: 18/963,972

(22) Filed: Nov. 29, 2024

Related U.S. Application Data
(60) Provisional application No. 63/604,226, filed on Nov. 30, 2023.

(51) Int. Cl. A61B 34/20; A61B 17/00

(52) U.S. Cl. CPC A61B 34/20; A61B 17/00; A61B 2017/00486; A61B 2034/2072

(57) ABSTRACT

An adapter mounts a tracking target to an end fitting of an existing r instrument. The adapter includes A shaft having an engagement fitting at one end thereof that is configured the same as the end fitting of the existing instrument so that a handle can be mounted on the adapter instead of on the end fitting of the tool/instrument. The adapter shaft includes a collet that cooperates with a locking nut to clamp the collet onto the instrument shaft when the end fitting of the instrument is disposed within the collet. A tracking element is provided that is detectable by a tracking system to determine the position and orientation of the instrument. The tracking element includes a mounting feature that is configured to snap onto the adapter shaft at a known axial position thereon while permitting rotation of the tracking element about the longitudinal axis of the adapter shaft.

### PRIORITY CLAIM

This application is a utility filing from and claims priority to U.S. Provisional Application No. 63/604,226, filed on Nov. 30, 2023, the entire disclosure of which is incorporated herein by reference.

### BACKGROUND

Image-guided procedures have become the technique of choice for certain types of surgeries. These procedures require knowledge of the location and orientation of instruments during the surgical procedure, which in turn requires precise tracking of the instruments in at least five, but preferably six, degrees of freedom.

Typical tracking systems are either optical or electromagnetic (EM). In either case, a tracking target is fastened to the surgical tool or instrument, and includes fiducials that can be detected by an optical or EM sensor. The fiducials are arranged on the tracking target in a manner that allows tracking software to determine the position of the tool or instrument relative to a global and/or local coordinate system.

Some companies that provide tracking systems also provide tools and instruments that are integrated with associated tracking targets. One example is shown in FIGS. 1-2 in which an instrument includes a target mount that includes a cylindrical body that is slid onto the instrument shaft. The instrument/tool includes an elongated shaft with a distal working end and a proximal end fitting. The working end can be any type of component for use in a surgical or diagnostic procedure, such a broach, implant trial, implant, bone screw, distraction wedge, etc. The proximal end fitting can be configured to engage a manipulation component grasped by the surgeon to manipulate the tool/instrument, such as a fixed or ratcheting handle, driver or T-handle. The end fitting is specifically configured to engage a mating fitting of the manipulation component engaged with the square head at the end of the instrument shaft in a conventional manner. Knowledge of the position and orientation of the working end during the procedure is critical for an image guided procedure using the tool/instrument. Thus, a tracking target is mounted on the target mount. The tracking target provides means for detection by a tracking system using optical or infrared detectors.

The instruments shown in FIGS. 1-2 are specifically configured to receive the cylindrical body of the target mount for use with a tracking system. However, not all tools and instruments are provided as part of a tracking system. In other words, not all instruments have a tracking target and target mount provided as an integrated assembly. Therefore, there is a need for an adaptor that allows integration of a tracking target onto any surgical tool or instrument.

Moreover, in some cases, the instrument must rotate about its axis, such as a driving tool for driving a bone screw into a vertebral body. In these cases, the fiducials of the tracking target must be visible to the optical or EM tracking sensor. Therefore, there is a further need for an adapter that allows the tracking target to be rotated about the tool or instrument so that the line of sight can be maintained between the fiducials and the tracking sensors.

### SUMMARY

An adapter is provided that mounts a tracking target to an end fitting of an existing tool or instrument. The end fitting of the tool/instrument is configured to engage the mating fitting of a manually-grasped component, such as a handle. The adapter includes an elongated adapter shaft having an engagement fitting at one end thereof that configured the same as the end fitting of the tool/instrument so that the manually-grasped component can be mounted on the adapter when the adapter is mounted on the end fitting of the tool/instrument. The adapter shaft further includes a collet at an opposite end thereof that cooperates with a locking nut to clamp the collet onto the instrument shaft when the end fitting of the instrument is disposed within the collet.

A tracking element is provided that includes a plurality of tracking components arranged on the body of the tracking element to be detected by a tracking system to determine the position and orientation of the tool/instrument when the adapter and tracking element are mounted on the tool/instrument. The tracking element includes a mounting feature that is configured to snap onto the adapter shaft at a known axial position thereon while permitting rotation of the tracking element about the longitudinal axis of the adapter shaft.

The adapter can be mounted on any tool with an end fitting, and a variety of tracking elements can be configured to snap onto the adapter shaft. The adapter is configured to be mounted on the end fitting of the tool/instrument and to provide a similar engagement fitting to receive the manually-grasped component for use of the tool/instrument. The tracking element can be rotated relative to the adapter and instrument shafts to maintain the tracking element in the line of sight of a tracking system, even when the tool/instrument is rotated in use.

### DESCRIPTION OF THE DRAWINGS

FIG. 1 is view of a prior art surgical instrument with a tracking target mounted thereon.

FIG. 2 is an exploded view of the components of the surgical instrument and tracking target shown in FIG. 1.

FIG. 3 is a perspective view of an adapter of the present disclosure.

FIG. 4 is a perspective view of a locking nut for the adapter of FIG. 3.

FIG. 5 is a side cross-sectional view of the adapter shown in FIG. 3.

FIG. 6 is a side view of the adapter shown in FIG. 3.

FIG. 7 is an end view of the adapter shown in FIG. 3.

FIG. 8 is a side cross-sectional view of the locking nut shown in FIG. 4.

FIG. 9 is an end view of the locking nut shown in FIG. 4.

FIG. 10 is a view of an exploded view of a surgical tool with the adapter of the present disclosure.

FIG. 11 is a view of a surgical tool with the adapter of the present disclosure mounted thereon and with a tracking target mounted to the adapter.

FIG. 12 is a view of the surgical tool with the adapter of the present disclosure mounted thereon, as shown in FIG. 11, with a handle mounted to the adapter.

FIG. 13 is a view of a surgical tool with a Hudson-type fitting.

FIG. 14 is a view of the surgical tool shown in FIG. 13 with a T-handle mounted to the Hudson-type fitting.

FIG. 15 is a view of the surgical tool shown in FIG. 13 with an adapter of the present disclosure that includes a Hudson-type fitting.

FIG. 16 is a view of the surgical tool and adapter shown in FIG. 15 with the T-handle mounted to the adapter.

FIG. 17 is a side view of a tracking target configured for mounting on the adapters of the present disclosure.

FIG. 18 is a top view of the tracking target shown in FIG. 17.

FIG. 19 is a partial view of a modified mounting component for use with a tracking target such as the tracking target shown in FIG. 17.

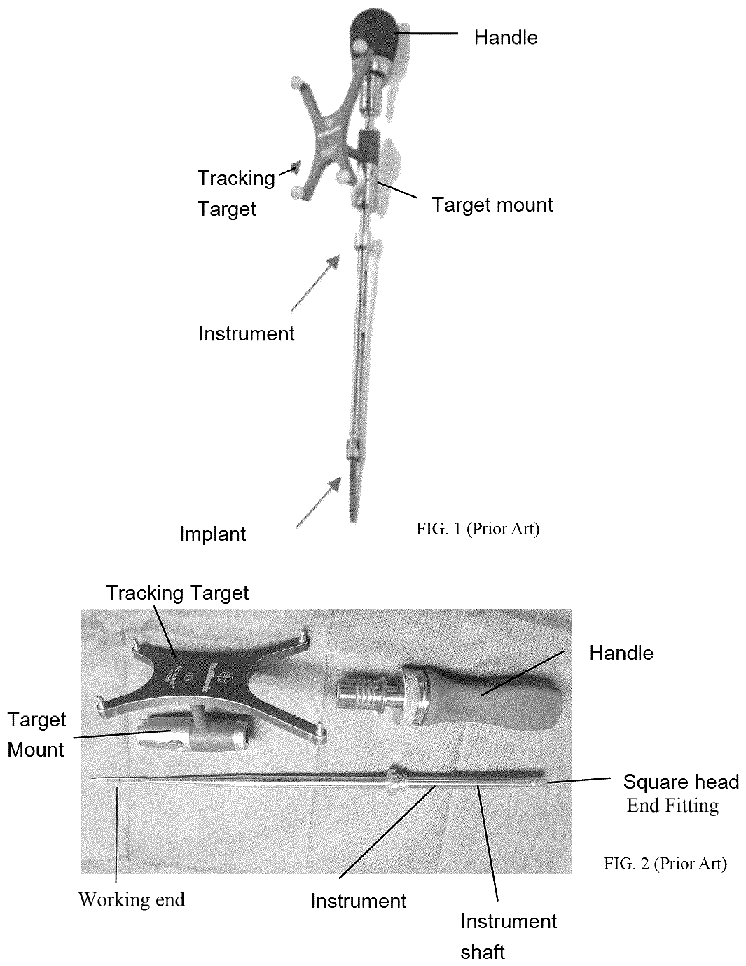

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles disclosed herein as would normally occur to one skilled in the art to which this disclosure pertains.

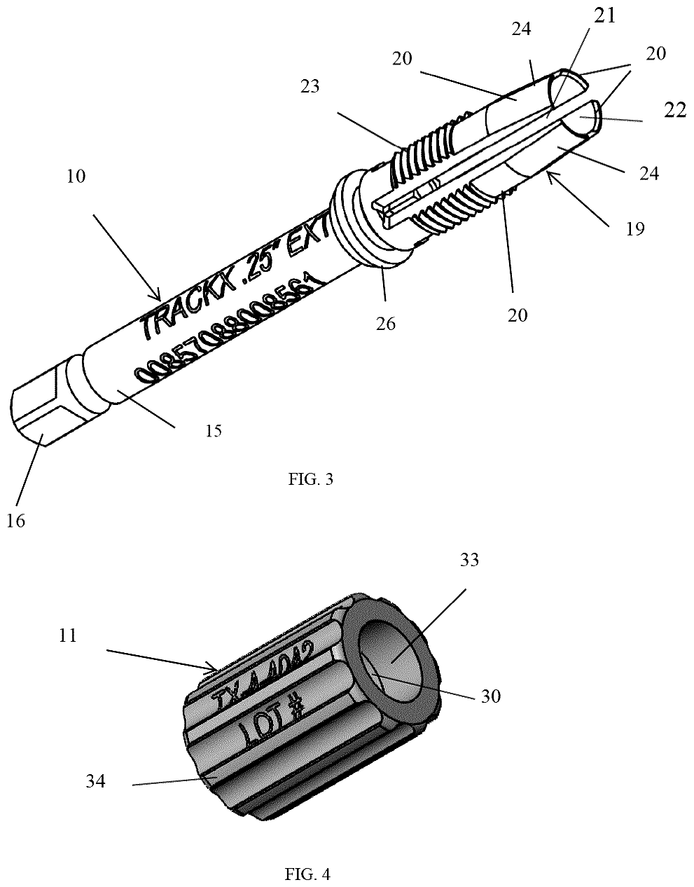

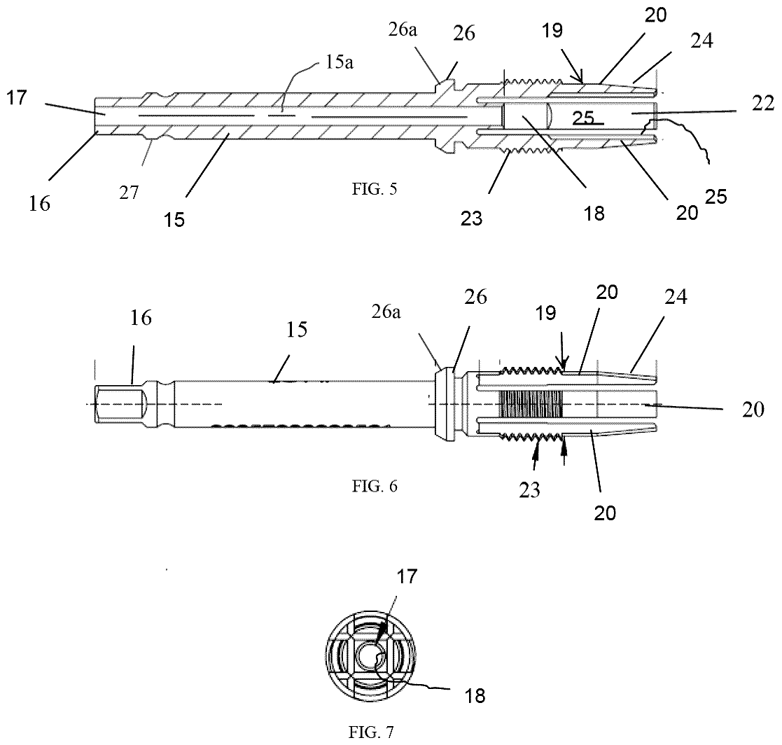

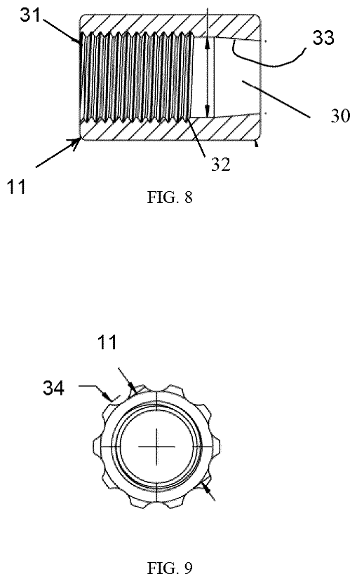

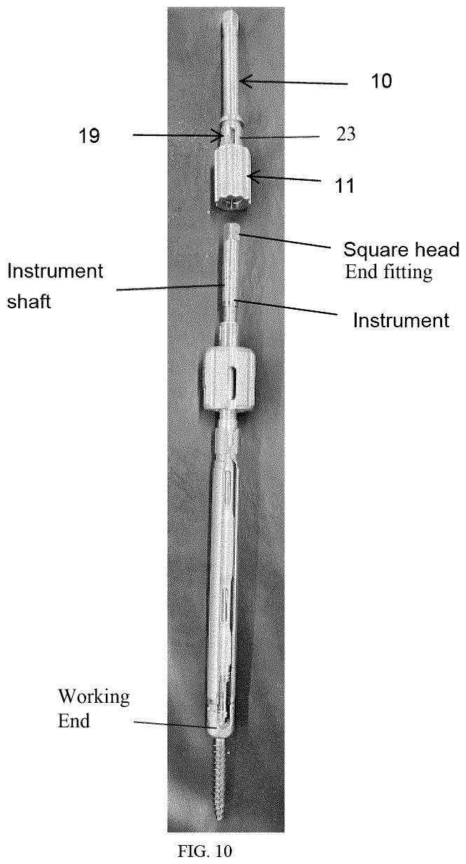

The present disclosure provides an adapter 10 and locking nut 11 that can be mounted on the end fitting of any surgical tool or instrument. The adapter 10, as shown in FIGS. 3-7, includes an elongated shaft 15 with an engagement head 16 at one end. The engagement head 16 has the same configuration as the end fitting of the tool/instrument on which the adapter is mounted. In the embodiment shown in FIG. 3, the engagement head 16 is a square fitting or socket to replace the square head/socket on the tool/instrument, such as the instrument shown in FIG. 10. The shaft 15 of the adapter can be cannulated with a bore 17 to receive other tools/instruments, such as a guide wire, that may otherwise be used with the tool/instrument. An interior end of the bore 17 defines an engagement bore 18 that is configured to engage the end fitting of the tool/instrument when the adapter 10 is mounted thereon. In the embodiment of FIG. 3, the engagement bore is square to engage the square end fitting of the tool/instrument. As described herein, the end fitting of the tool/instrument can be a square or hex fitting, a Hudson-type fitting, or other fittings that are used to engage a component to the end of the tool/instrument. The engagement head 16 of the adapter 10 has the same configuration as the end fitting at the proximal end of the tool/instrument. Likewise, the engagement bore 18 is configured to receive the particular end-fitting of the tool/instrument.

The shaft 15 includes a collet 19 at the opposite end of the shaft, as shown in detail in FIGS. 5-6. The collet 19 includes a plurality of wings 20 that are separated by gaps 21 and define a bore 22 sized to fit over the end fitting and shaft of the tool/instrument. The collet wings 20 are configured to deflect toward each other and inward into the bore 22. The wings define an interior clamping surface 25 facing the bore 22. One end of the wings adjacent the shaft 15 includes exterior threads 23 while the opposite free end of the wings includes an inwardly tapered exterior surface 24.

The locking nut 11, as shown in FIGS. 4, 8, 9, defines a bore 30 with internal threads 31 at one end and an internal tapered portion 33 at the opposite end. The internal threads 31 are configured for threaded engagement with the external threads 23 of the collet. Similarly, the internal tapered portion 33 of the locking nut 11 is configured to bear against the tapered exterior surfaces 24 of the collet wings 19 as the nut is threaded onto the threads of the collet. In particular, the two complementary tapered portions are configured so that the interior clamping surface 25 narrows onto the shaft of the tool/instrument as the locking nut 11 is threaded further onto the collet. A thread stop 32 at the end of the internal threads 31 of the nut 11 is configured to limit the amount that the nut can be threaded onto the collet. The nut is provided with an external manual engagement feature 34 (FIG. 9) to facilitate manual rotation of the locking nut 11 on the adapter 10.

As shown in FIG. 10, the nut 11 can be loosely threaded onto the collet threads 23 to hold the nut on the adapter 10 as the adapter is mounted on the end of the tool/instrument. The entire assembly is advanced onto the instrument shaft until the end fitting of the instrument shaft (in this case a square head) is disposed within the engagement bore 18 of the adapter 10. In the case of a square head end fitting, the square configuration of the engagement bore locks the adapter in rotation with the instrument shaft when the instrument end fitting is disposed within the engagement bore 18. The locking nut 11 can be rotated to further engage the collet threads 23 until the collet wings 20 are clamped onto the instrument shaft.

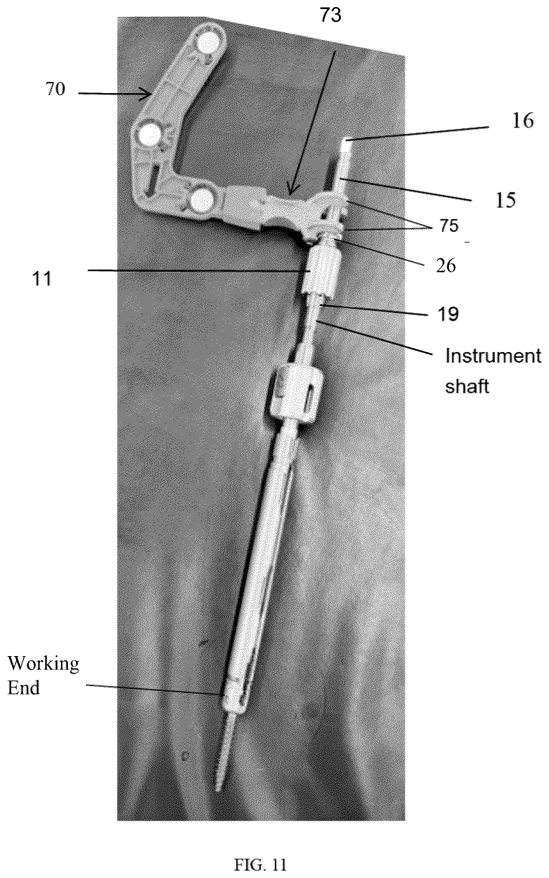

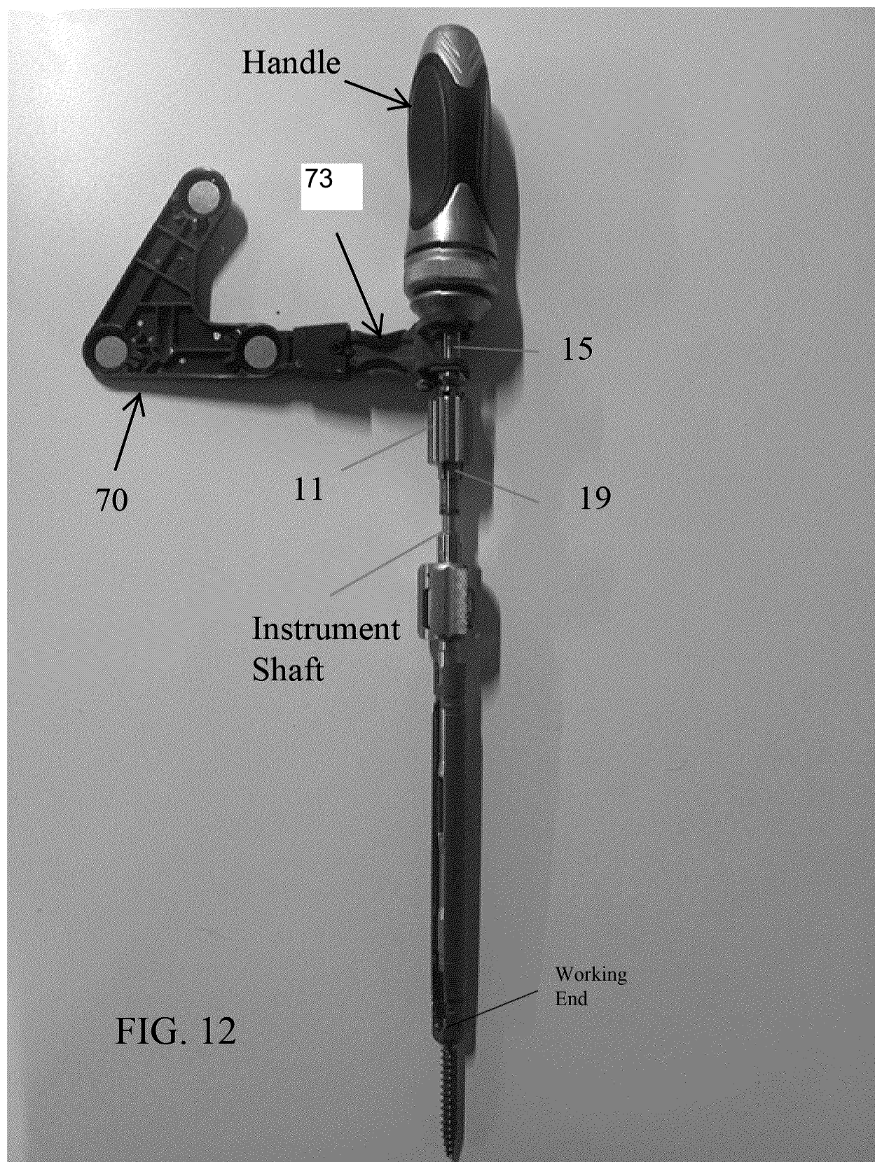

With the adapter 10 clamped onto the end of the instrument shaft, the mounting feature 72 of a tracking element 70 can be engaged to the shaft 15 of the adapter 10. In one aspect of the present disclosure, the mounting feature 72 is configured to resiliently snap or clip onto the shaft 15 to hold the axial position of the tracking element while permitting relative rotation of the tracking element 70 about the longitudinal axis 15a of the adapter shaft. This allows the surgeon to adjust the position of the tracking element to ensure a line of sight to the sensors of the tracking system, even as the tool/instrument is manipulated and rotated. An enlarged circumferential shoulder 26 at the distal end of the shaft adjacent the collet 19 fixes the axial position of the tracking element on the adapter, as shown in FIGS. 11-12. The shoulder 26 can have a tapered or rounded surface 26a to engage a complementary tapered or rounded surface of the mounting feature of the tracking element. The flange establishes a known axial position of the tracking element relative to the working end of the tool/instrument. This feature is important for image-guided procedures since it is critical that the position of the working tip be accurately known.

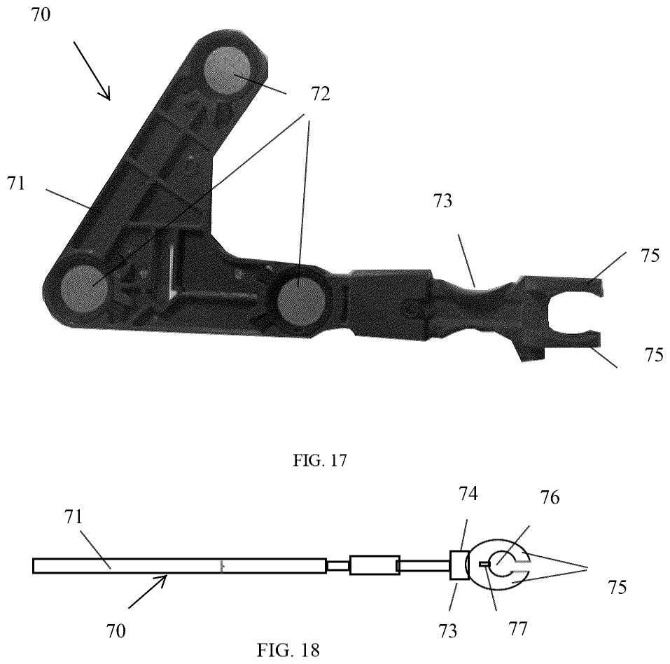

Details of the tracking element 70 are shown in FIGS. 17-19. The element 70 includes a body 71 that supports tracking components 72 that are configured to be detected by an image localization or tracking system use to determine the position and orientation of the tool/instrument in a surgical site. For instance, the tracking components can be configured for optical or IR detection. In the tracking element 70, three tracking components 72 can be provided that can be used to accurately determine the spatial orientation of the instrument in 5 or 6 degrees of freedom. The mounting feature 73 is integral with or attached to the body 71 of the adapter. The mounting feature of FIGS. 17-18 includes a base 74 from which two pairs of resilient clips 75 extend. As shown in FIG. 18, the claims define a central bore 76 sized to snap or clip into engagement with the shaft 15 of the adapter 10. A notch 77 at the base of the central bore allows the clips 75 to resiliently deflect outward as the mounting feature is pressed onto the shaft 15. The central bore 76 is sized to provide some frictional engagement with the shaft, while still permitting the tracking element 70 to be rotated relative to the axis 15a of the adapter shaft. As shown in FIG. 17, the two pairs of resilient clips 75 are offset from each other to provide two points of engagement with the shaft 15, as shown in FIG. 11. The outboard faces of each of the resilient clips can define a tapered or rounded surface to seat on the shoulder 26 to establish a known axial position of the tracking element 70 on the instrument shaft.

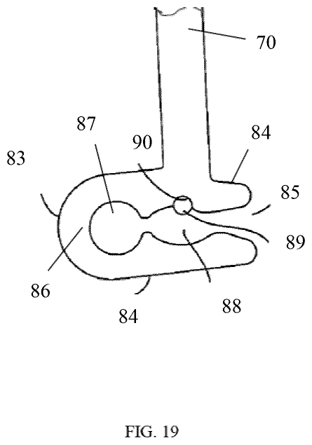

In another embodiment, the tracking element 70 can be provided with the mounting feature 83 shown in FIG. 19. This mounting feature 83 corresponds to the mounting portion 502 of the tracking element 500 shown in FIGS. 43, 44, 48-50 of U.S. Pat. No. 11,612,441 (the '441 Patent), which issued to the present application on Mar. 28, 2023. These figures and the corresponding description at col. 21, In. 27-col. 24, ln. 6, are specifically incorporated herein by reference. As disclosed in the '441 Patent, the mounting feature 83 includes a pair of legs 84 that define a channel 85 between the legs. The legs 84 are resiliently deflectable, with the end 86 and circular bore 87 of the feature acting as a resilient hinge. The legs further define a generally circular cavity 88 that is sized to receive the shaft 15 of the adapter 10 with sufficient friction to hold the tracking element 70 on the shaft while permitting rotation of the tracking element relative to the adapter 10 and the tool/instrument on which the adapter is mounted. In one embodiment, a friction peg 89 can be disposed within a notch 90 in one side of the circular cavity 88. The friction peg 89 can enhance the grip of the mounting feature 83 on the adapter shaft 15, as described in the '441 Patent.

Other configurations of the tracking element 70 can be implemented with the adapter 10 disclosed herein. The '441 Patent discloses alternative tracking elements 45 (FIG. 9), 400 (FIG. 38), 420 (FIGS. 39A-B), 500 (FIG. 41), 500′ (FIG. 45). 500″ (FIG. 46), 540 (FIG. 47), 610 (FIG. 52), 636 (FIGS. 56A-B), and 647 (FIGS. 58A-B), each of which can be implemented with the adapter 10. Details of the alternative mounting features and tracking elements disclosed in the '441 Patent are incorporated herein by reference.

It should be appreciated that the engagement head 16 of the adapter 10 emulates the fitting at the end of the instrument onto which the adapter is mounted. Thus, in the embodiment of FIGS. 11, the engagement head 16 is a square head so that a handle can be engaged to the adapter to rotate the entire assembly, as shown in FIG. 12. A circumferential flange 27 is provided below the engagement head to help fix the axial position of the handle or other component when it is mounted on the engagement head 16.

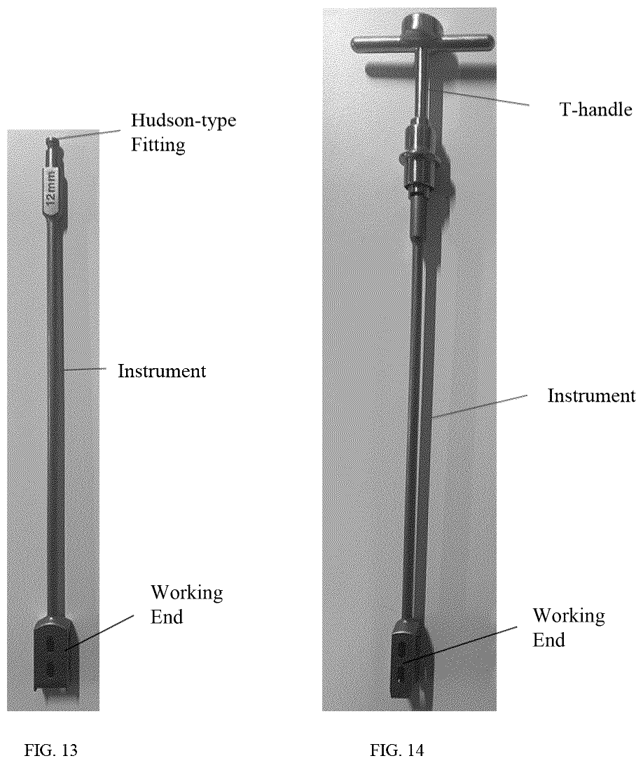

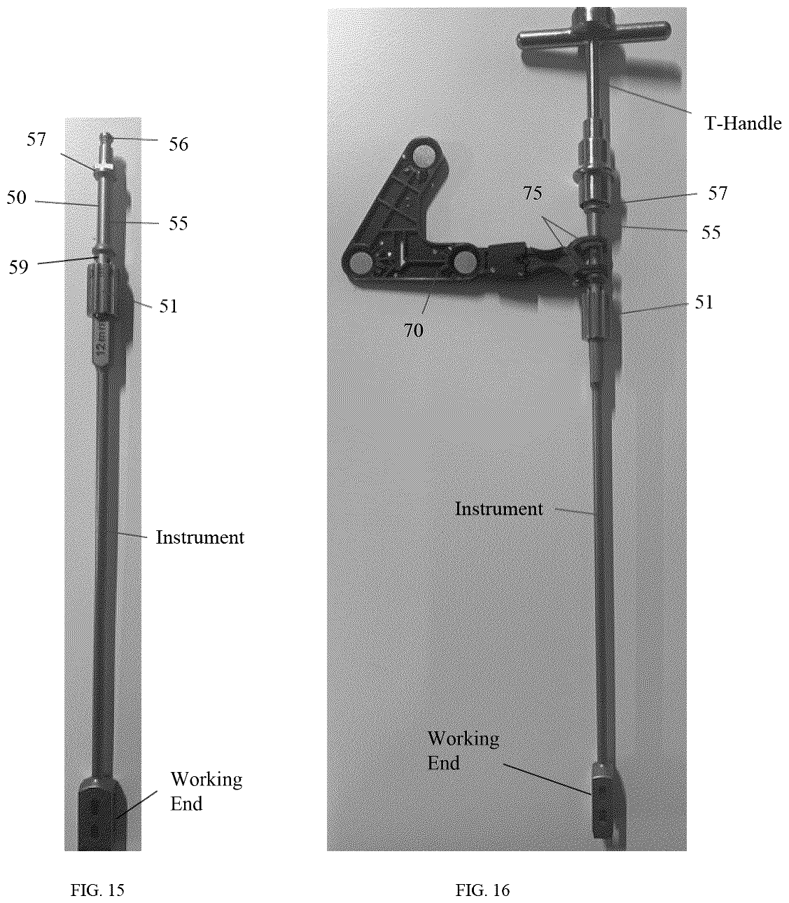

The square head of the tool/instrument shown in the examples is one of several fitting configurations. Other fittings include the Hudson and the AO fittings. The adapter 10 of the present disclosure can be modified to accommodate other fitting configurations. In particular, the engagement bore 18 of the adapter can be modified to accept the particular fitting. Likewise, the engagement head 16 of the adapter can be modified to the same fitting configuration as the tool/instrument. As shown in FIG. 13, an instrument includes a Hudson-type fitting at its proximal end that is configured to engage a T-handle, as shown in FIG. 14. The adapter 50 shown in FIG. 15-16 includes a shaft 55, collet 59 and locking nut 51 having the same configuration as the shaft 15 and nut 11 of the previous embodiment. However, the engagement head 56 of the adapter 50 differs from the engagement head 16 in that the head 56 is configured as the Hudson-type fitting of the instrument, so that the T-handle can be mounted on the adapter 50, as shown in FIG. 16. Although not shown in FIGS. 15-16, the adapter includes an engagement bore, similar to the engagement bore 18 except configured to receive the Hudson-type fitting of the instrument shown in FIG. 13. It is understood that the interface between the Hudson-type fitting and complementary engagement bore will not fix the adapter in rotation relative to the shaft of the instrument, as is the case with the square head fitting of the previous embodiment.

In one specific embodiment, the adapter 10 can have a length of 3.742 in., with the collet 19 having a length of 1.181 in. The bore 22 of the collet has a depth of 1.015 in. to the engagement bore 18. Thus, the shaft 15 of the adapter has a length of 2.727 in. from the end of the engagement head 16 to the engagement bore 18. It can thus be appreciated that the adapter will overlap the existing instrument shaft by the depth of the bore 22 and extend axially beyond the end of the instrument shaft by 2.727 in. with this specific embodiment. The bore 22 of the collet 19 has a diameter of 0.315 in. to receive a standard instrument shaft. The complementary angles between the exterior tapered surface 24 of the collet 19 and the tapered portion 33 of the locking nut can be 8-10 degrees. The angle of the tapered portions can be configured for self-locking engagement, such as with a 17.5-20 degree included angle.

The mounting features 73, 83 are configured so that the tracking element 70 can be snapped onto the shaft 15 of the adapter 10 at any time, but particularly after the adapter and additional component (such as the handle of FIG. 12 or the T-handle of FIG. 16) have been mounted on the particular instrument. The adapter 10 disclosed herein allows for concentric mating to any tool/instrument to support the tracking element at a known fixed distance from the working tip of the tracked tool/instrument. A different adapter is provided for each type of end fitting of the tools/instruments to be used in the image guided procedure. The selection of adapters can be otherwise identical.

It is contemplated relationship between the tracking element 70 and the working end of the tool/instrument can be established in a calibration procedure prior to use of the tool/instrument in a procedure. The calibration process will establish the axial and radial distance of the tracking components 72 from the working tip of the tool/instrument, which data can be provided to the tracking system. Tracking system software can then determine the global and/or local position of the working tip when the global/local positions of the tracking components are established. The adapter 10 thus allows any tool/instrument to be used in an image-guided procedure.

The present disclosure should be considered as illustrative and not restrictive in character. It is understood that only certain embodiments have been presented and that all changes, modifications and further applications that come within the spirit of the disclosure are desired to be protected.

What is claimed is:

1. An assembly for mounting on a tool/instrument having an elongated instrument shaft with a distal working end and a proximal end fitting, the end fitting configured to engage the mating fitting of a manually-grasped component, the assembly comprising:
an adapter including;
an elongated adapter shaft having an engagement fitting at one end thereof configured the same as the end fitting of the tool/instrument for removable engagement with the mating fitting of the manually-grasped component;
said adapter shaft including a collet at an opposite end thereof, said collet including a plurality of wings defining an elongated bore for receiving the end fitting and a proximal portion of the instrument shaft, said plurality of wings including interior clamping surfaces and configured to deflect toward each other into said bore so that said interior clamping surfaces engage the instrument shaft, said plurality of wings further including exterior threads at an end of said wings adjacent said adapter shaft and exterior tapered surfaces at an opposite end of said wings; and
a locking nut having internal threads configured for threaded engagement with said exterior threads of said collet, and an internal tapered portion configured to bear against said exterior tapered surfaces of said plurality of wings to deflect said wings inward with said interior clamping surfaces in clamping engagement with the instrument shaft; and
a tracking element including;
a body supporting a plurality of tracking components arranged on said body to be detected by a tracking system to determine the position and orientation of the tool/instrument when the apparatus is mounted on the tool/instrument;
a mounting feature attached to said body, said mounting feature configured to snap onto said adapter shaft at a known axial position thereon while permitting rotation of said tracking element about the longitudinal axis of said adapter shaft.

2. The assembly of claim 1, wherein said adapter shaft is cannulated.

3. The assembly of claim 1 in which the end fitting of the instrument and said engagement fitting are a square fitting.

4. The assembly of claim 1 in which the end fitting of the instrument and said engagement fitting are a Hudson-type fitting.

5. The assembly of claim 1, wherein said adapter shaft includes an enlarged shoulder and said mounting feature defines a complementary face to seat on said enlarged shoulder when said mounting feature is snapped onto said adapter shaft to fix the axial position of said tracking element relative to the working end of the tool/instrument.

6. The assembly of claim 1, wherein said mounting feature includes a pair of resilient clips configured to resiliently engage said adapter shaft with said resilient clips spaced apart along the longitudinal axis of said adapter shaft.

7. The assembly of claim 1, wherein said elongated bore of said collet defines an engagement bore adjacent said shaft, said engagement bore configured to receive the end fitting of said tool/instrument when the instrument shaft is received within said elongated bore.

8. The assembly of claim 7 in which the end fitting of the instrument and said engagement fitting are each a square fitting, and said engagement bore is configured for complementary engagement with a square fitting.

9. The assembly of claim 7 in which the end fitting of the instrument and said engagement fitting are a Hudson-type fitting, and said engagement bore is configured for engagement with a Hudson-type fitting.

10. The assembly of claim 1, wherein said shaft includes a circumferential flange adjacent said engagement fitting for engaging the manually-grasped component to fix the axial position of the manually-grasped component on said engagement fitting.

11. An assembly comprising:
a manually-grasped component for manipulating a tool/instrument;
a tool/instrument having an elongated instrument shaft with a distal working end and a proximal end fitting, the end fitting configured to engage a mating fitting of the manually-grasped component;
an adapter including;
an elongated adapter shaft having an engagement fitting at one end thereof configured the same as the end fitting of the tool/instrument for removable engagement with the mating fitting of the manually-grasped component;
said adapter shaft including a collet at an opposite end thereof, said collet including a plurality of wings defining an elongated bore for receiving the end fitting and a proximal portion of the instrument shaft, said plurality of wings including interior clamping surfaces and configured to deflect toward each other into said bore so that said interior clamping surfaces engage the instrument shaft, said plurality of wings further including exterior threads at an end of said wings adjacent said adapter shaft and exterior tapered surfaces at an opposite end of said wings; and
a locking nut having internal threads configured for threaded engagement with said exterior threads of said collet, and an internal tapered portion configured to bear against said exterior tapered surfaces of said plurality of wings to deflect said wings inward with said interior clamping surfaces in clamping engagement with the instrument shaft; and
a tracking element including;
a body supporting a plurality of tracking components arranged on said body to be detected by a tracking system to determine the position and orientation of the tool/instrument when the apparatus is mounted on the tool/instrument;
a mounting feature attached to said body, said mounting feature configured to snap onto said adapter shaft at a known axial position thereon while permitting rotation of said tracking element about the longitudinal axis of said adapter shaft.

12. The assembly of claim 11, wherein said adapter shaft is cannulated.

13. The assembly of claim 11, wherein said end fitting of the instrument and said engagement fitting are a square fitting.

14. The assembly of claim 11, wherein the end fitting of the instrument and said engagement fitting are a Hudson-type fitting.

15. The assembly of claim 11, wherein said adapter shaft includes an enlarged shoulder and said mounting feature defines a complementary face to seat on said enlarged shoulder when said mounting feature is snapped onto said adapter shaft to fix the axial position of said tracking element relative to said working end of said tool/instrument.

16. The assembly of claim 11, wherein said mounting feature includes a pair of resilient clips configured to resiliently engage said adapter shaft with said resilient clips spaced apart along the longitudinal axis of said adapter shaft.

17. The assembly of claim 11, wherein said elongated bore of said collet defines an engagement bore adjacent said shaft, said engagement bore configured to receive said end fitting of said tool/instrument when the instrument shaft is received within said elongated bore.

18. The assembly of claim 17, wherein said end fitting of the instrument and said engagement fitting are a square fitting, and said engagement bore is configured for complementary engagement with a square fitting.

19. The assembly of claim 17, wherein said end fitting of the instrument and said engagement fitting are a Hudson-type fitting, and said engagement bore is configured for engagement with a Hudson-type fitting.

20. The assembly of claim 11, wherein said shaft includes a circumferential flange adjacent said engagement fitting for engaging the manually-grasped component to fix the axial position of the manually-grasped component on said engagement fitting.
